(19) United States
(12) Patent Application Publication
(10) Pub. No.: US 2024/0091549 A1
(43) Pub. Date: Mar. 21, 2024

(54) WEARABLE REPELLING MAGNET ASSEMBLY

(71) Applicant: Hirofusa Otsubo, Middle Village, NY (US)

(72) Inventor: Hirofusa Otsubo, Middle Village, NY (US)

(21) Appl. No.: 17/896,266

(22) Filed: Aug. 26, 2022

(51) Int. Cl. A61N 2/06; H01F 7/02

(52) U.S. Cl. CPC A61N 2/06; H01F 7/0205

(57) ABSTRACT

A wearable repelling magnet assembly comprising a plurality of magnetic components and a retaining member, the retaining member forms a physical linkage between the magnetic components. The magnetic components each project a magnetic field and form a repelling sequence in which each magnetic component exerts a repelling force against the adjacent magnetic components within the sequence, creating a separation interval between the magnetic components and further redirecting the magnetic fields outwardly away from a magnetic axis. The retaining member may be configured as a loop to be worn about a wearer's body.

### TECHNICAL FIELD

The present disclosure relates generally to a wearable therapeutic device. More particularly, the present disclosure relates to a wearable apparatus containing a sequence of magnetic components held together in a repelling configuration.

### BACKGROUND

Magnets are commonly used to provide homeopathic treatments for chronic disease. Magnetic fields induce electrons to flow in a negatively charged wave and have been demonstrated to raise pH levels in water towards alkalinity. Magnetic fields can therefore influence the flow of free electrons to alter voltage potential within cells of the human body and stimulate healing.

Many magnetic therapeutic devices can be found within the prior art, particularly wearable devices which are held in close proximity to or against the body. Many such devices contain magnets linked together by magnetic attraction. However, magnetic attraction causes magnetic force lines to flow between magnets, thus narrowing areas around the device where magnetic exposure is possible. Other devices employ a spread of standard magnets which project unaltered magnetic fields which flow from pole to pole. These devices do not provide sufficient magnetic exposure, and the resulting magnetic fields are weak.

However, the devices within the prior art do not employ magnets placed in a repelling configuration in which the magnetic fields are redirected outwardly away from a magnetic axis by repelling magnetic forces. Such an arrangement would widen the areas around a device in which magnetic exposure is possible, and would further strengthen the magnetic repelling forces produced by placing repelling magnets in very close proximity with each other.

In contrast to the devices within the prior art, repelling magnets can be used to apply fundamental principles of quantum medicine to promote health and healing. It is commonly known that two properly configured magnets placed in a repelling position will create a demagnetization effect resulting in a significant reduction of magnetic strength caused by the collision of repelling magnetic fields. The collision of repelling magnetic fields alters the flow of electrons within the magnetic fields and dislodges some of the electrons from their normal flow, turning them into free electrons. Positioning repelling magnets near the human body thus exposes the body to a supply of free electrons which act as electron donors to restore the pH of the body to a healthy level of alkalinity.

Therefore, there is a clear and unfulfilled need for a new wearable magnetic device which employs a dense array of repelling magnets to produce strong repelling forces which increase exposure of free electrons to a wearer, thus enhancing the beneficial effects of magnetic therapy according to the principles of quantum medicine.

In the present disclosure, where a document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

An aspect of an example embodiment in the present disclosure is to provide a wearable magnetic apparatus for exposing a wearer to free electrons. Accordingly, the present disclosure provides a repelling magnetic assembly comprising a plurality of magnetic components and a retaining member. Each magnetic component has a channel and projects a magnetic field, and the retaining member extends through the channels of the magnetic components to form a physical linkage therebetween. Furthermore, the retaining member may be formed as a loop or segment which can be placed in close proximity to a wearer's body or around a portion thereof.

It is an aspect of an example embodiment in the present disclosure to provide a wearable magnetic apparatus which projects a concentration of magnetic fields which project outwardly to increase magnetic exposure. Accordingly, the plurality of magnetic components form a repelling sequence disposed along the retaining member in which each of the magnetic components exerts a magnetic repelling force against each magnetic component adjacent to it within the repelling sequence. Furthermore, the repelling forces act upon the magnetic fields to spread and redirect the magnetic force lines outwardly away from a magnetic axis, thus increasing magnetic exposure to the wearer.

It is a further aspect of an example embodiment in the present disclosure to provide a wearable magnetic apparatus which increases the redirection of the magnetic field by strengthening the repelling forces. The strength of the magnetic repelling force is inversely proportional to the separation interval between two repelling magnets. Accordingly, the repelling sequence can be formed using a plurality of alternate magnetic components each comprising two repelling magnetic subcomponents forced together in physical contact, thus minimizing the separation interval. Direct physical contact between the two magnetic subcomponents is maintained using a bonding means such as a bonding adhesive, or by encasing the two magnetic subcomponents within a containing shell.

The present disclosure addresses at least one of the foregoing disadvantages and solutions. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### BRIEF DESCRIPTION OF THE DRAWINGS

In the drawings, like elements are depicted by like reference numerals. The drawings are briefly described as follows.

FIG. 1A is a diagrammatical top view of a repelling magnet assembly containing a repelling sequence of magnetic components held together in a repelling configuration by a retaining member, in accordance with an embodiment in the present disclosure.

FIG. 1B is a diagrammatical perspective view of the repelling magnet assembly being worn around a wearer's wrist, in accordance with an embodiment in the present disclosure.

FIG. 2A is diagrammatical perspective view of one of the magnetic components, showing a channel which passes between a component first face and a component second face of the magnetic component, in accordance with an embodiment in the present disclosure.

FIG. 2B is a diagrammatical perspective view of an alternative magnetic component formed of two magnetic subcomponents affixed together in the repelling configuration, in accordance with an embodiment in the present disclosure.

FIG. 3A is a side sectional view of the retaining member passing through the channel of one of the magnetic components, in accordance with an embodiment in the present disclosure.

FIG. 3B is a side view of a repelling sequence of magnetic components oriented in a repelling configuration, where magnetic repelling forces maintain separation intervals between each of the magnetic components within the repelling sequence, in accordance with an embodiment in the present disclosure.

FIG. 4A is a diagrammatical depiction of the magnetic fields projected by two magnetic components oriented in the repelling configuration, the magnetic fields are depicted using field lines, in accordance with an embodiment in the present disclosure.

FIG. 4B is a diagrammatical depiction of the magnetic fields of the two magnetic components influencing free electrons present within the magnetic fields, in accordance with an embodiment in the present disclosure.

FIG. 4C is a diagrammatical top view of the repelling magnetic assembly worn around a representation of the wrist, further showing the magnetic field lines flowing into a loop interior space resulting from the repelling forces exerted by the magnetic fields of the magnetic components, in accordance with an embodiment in the present disclosure.

FIG. 5A is a diagrammatical side view of a sequence of alternate magnetic components, in accordance with an embodiment in the present disclosure.

FIG. 5B is a side sectional view of two repelling magnetic subcomponents held in contact by an adhesive bonding means, in accordance with an embodiment in the present disclosure.

FIG. 5C is a side sectional view of two magnetic subcomponents held in contact in the repelling configuration within a containing shell, in accordance with an embodiment in the present disclosure.

FIG. 6A is a diagrammatical depiction of a variation of the repelling magnet assembly in a segment configuration where the magnetic components are positioned between a first end and a second end of the retaining member, in accordance with an embodiment in the present disclosure.

FIG. 6B is a diagrammatical top view of a variation of the retaining member configured with a fastener which allows the first end and the second end to be detachably fastened therebetween, in accordance with an embodiment in the present disclosure.

FIG. 7A is a diagrammatical perspective view of a variant of the magnetic component configured as an elongated cylinder, in accordance with an embodiment in the present disclosure.

FIG. 7B is a diagrammatical front view of a variant of the magnetic component with the channel opening in a rectangular configuration, in accordance with an embodiment in the present disclosure.

FIG. 7C is a diagrammatical perspective view of a variant of the magnetic component configured as a rectangular prism, in accordance with an embodiment in the present disclosure.

FIG. 8A is a diagrammatical perspective view of the repelling magnet assembly employed with a liquid container, in accordance with an embodiment in the present disclosure.

FIG. 8B is a diagrammatical top view of the repelling magnet assembly surrounding the liquid container, further showing the container interior holding a liquid, in accordance with an embodiment in the present disclosure.

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete and fully conveys the scope of the present disclosure to those skilled in the art.

### DETAILED DESCRIPTION OF THE PREFERRED EMBODIMENTS

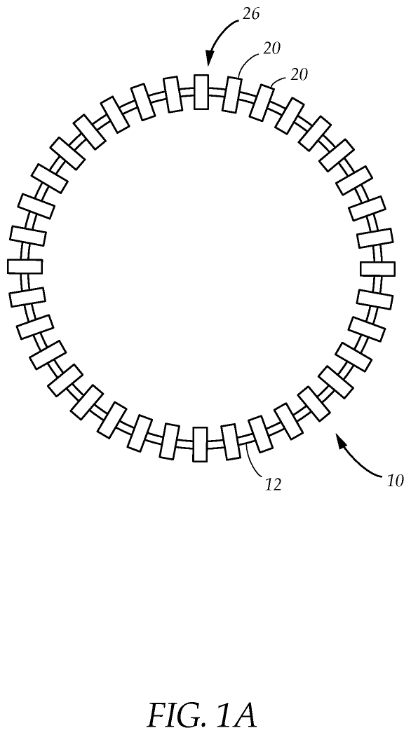

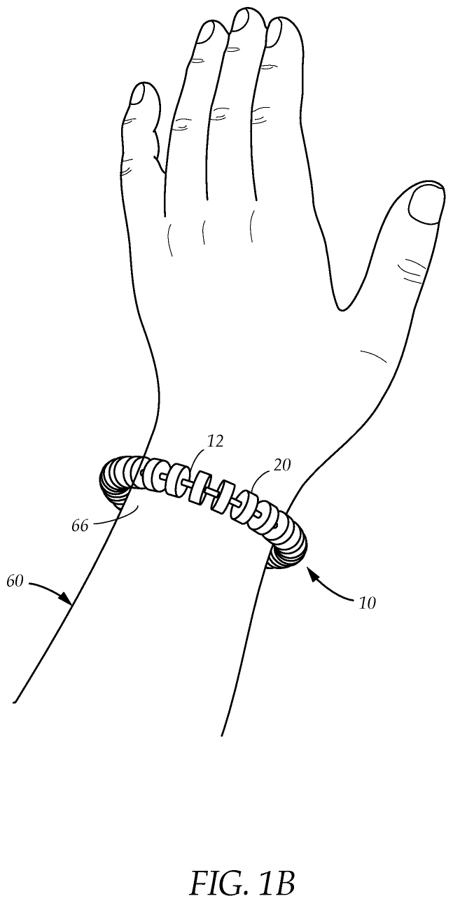

FIGS. 1A-B illustrate a repelling magnet assembly 10 comprising a retaining member 12 and a plurality of magnetic components 20. The plurality of magnetic components 20 are arranged in a repelling sequence 26 comprising at least two of the magnetic components 20 disposed in a linear fashion, in which each of the magnetic components 20 magnetically repels the magnetic components 20 adjacent thereto. Each of the magnetic components 20 is secured within the repelling sequence 26 by the retaining member 12. By varying the structure and shape of the retaining member 12, the repelling magnet assembly 10 may be implemented in a variety of configurations which allow it to be worn by a wearer 60. As contemplated in various embodiments, the repelling magnet assembly 10 may be placed adjacent to or in close proximity to the wearer's 60 body, or be wrapped around a portion of the body. For example, the retaining member 12 may be configured as a loop which allows the repelling magnet assembly 10 to be worn around the wrist 66 of the wearer 60.

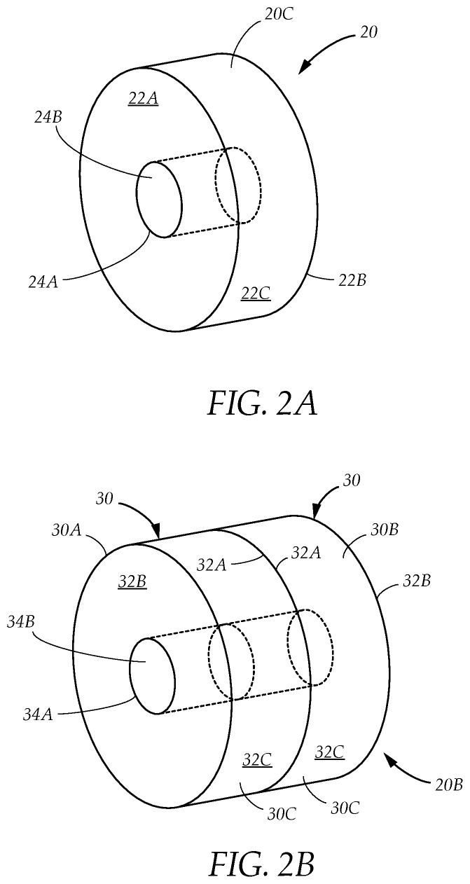

Referring to FIG. 2A while continuing to refer to FIGS. 1A-B, each magnetic component 20 has a component first face 22A, a component second face 22B oriented distally in relation to the component first face 22B, and a component body 20C which extends between the component first face 22A and the component second face 22B. Each magnetic component 20 contains ferromagnetic metals such as iron, cobalt, nickel, or rare earth metals, an may be configured in a variety of shapes. For example, the magnetic component 20 may be configured as a disc with the component body 20C having a cylindrical component outer surface 22C extending between the component first face 22A and the component second face 22B. In one embodiment, the component first face 22A and the component second face 22B are substantially flat and are disposed in a transverse manner in relation to the component outer surface 20C.

Each magnetic component 20 has a pair of channel openings 24A positioned on the component first face 22A and the component second face 22B, and a channel 24B which passes through the component body 20C between the pair of channel openings 24A. In a preferred embodiment, the channel openings 24A are disposed centrally upon the component first face 22A and the component second face 22B, and the channel 24B passes longitudinally though the component body 20C.

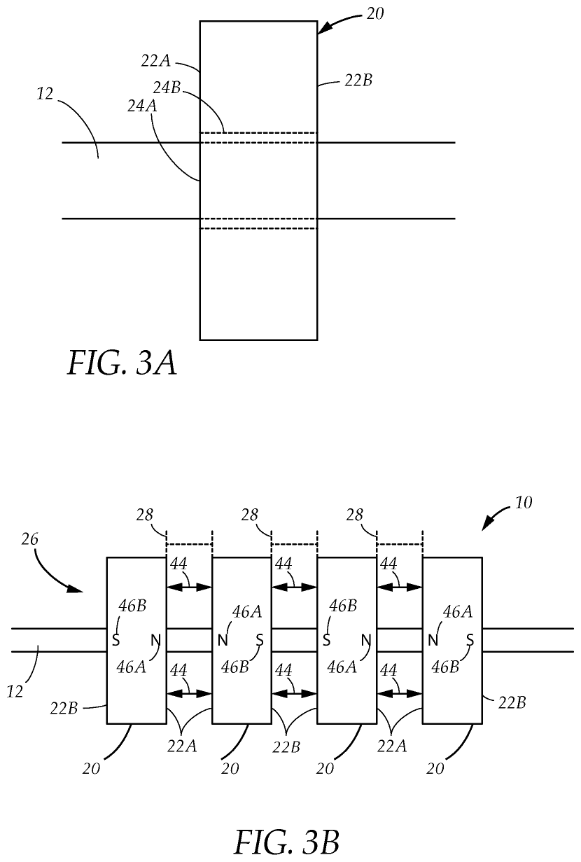

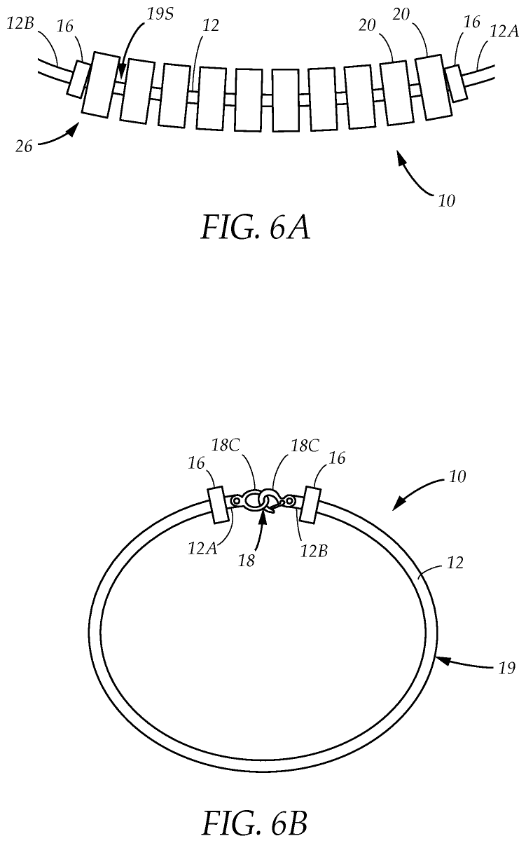

Referring to FIG. 3A while also referring to FIG. 1A and FIGS. 6A-B, the retaining member 12 provides a physical linkage which secures each of the magnetic components 20 within the repelling sequence 26. In a preferred embodiment, the retaining member 12 has a length and a width, with the length being substantially greater than the width. For example, the retaining member 12 may be formed as a rod, a wire, a chain, a tube, or other structure in which the length of the structure greatly exceeds the width. In turn, the width of the retaining member 12 is narrower than a diameter of the channel opening 24A and the channel 24B of each of the magnetic components 20. The retaining member 12 passes longitudinally through the channel 24B of each of the magnetic components 20 within the repelling sequence 26.

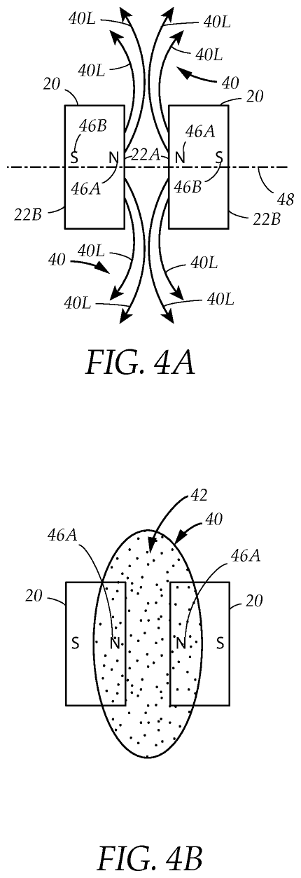

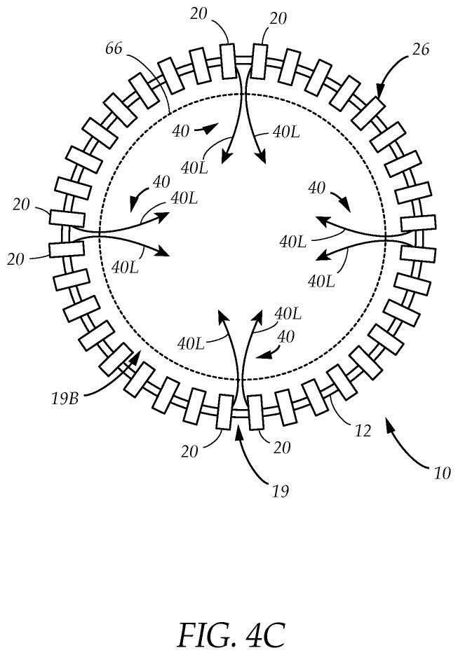

Turning to FIGS. 3A-B, FIG. 4A, and FIG. 4C while also referring to FIG. 1A and FIG. 2A, each magnetic component 20 projects a magnetic field 40. The magnetic components 20 within the repelling sequence 26 are disposed in a repelling configuration, in which each of the magnetic components 20 exerts a magnetic repelling force 44 against the magnetic components 20 adjacent thereto and creates a separation interval 28 between each of the magnetic components 20 within the repelling sequence 26. In a preferred embodiment, the repelling force 44 is sufficient to prevent the magnetic components 20 from sliding or being displaced along the retaining member 12 when exposed to moderate physical force, thus avoiding the need to physically adhere or bond the channel 24B to the retaining member 12.

In one embodiment, each magnetic component 20 has a first magnetic pole 46A oriented with the component first face 22A and a second magnetic pole 46B oriented with the component second face 22B. Each magnetic component 20 may have a magnetic axis 48 which extends longitudinally between the component first face 22A and the component second face 22B and which may be approximately coaxial with the channel 24B. The first magnetic pole 46A and the second magnetic pole 46B have opposite magnetic polarities. Opposite magnetic polarities will cause a magnetic attractive force to be exerted, whereas like magnetic polarities will cause a repelling force 44 to be exerted. For illustrative purposes, the first magnetic pole 46A may correspond to the north pole, while the second magnetic pole 46B may correspond to the south pole.

The repelling configuration ensures that each magnetic component 20 within the repelling sequence 26 will exert a repelling force 44 against each magnetic component 20 to which it is adjacent. In one embodiment, the magnetic components 20 are arranged such that the component first face 22A of each magnetic component 20 is oriented towards the component first face 22A of the magnetic component adjacent thereto. Conversely, the component second face 22B of each magnetic component 20 is oriented towards the component second face 22B of the adjoining magnetic component 20.

For illustrative purposes, an example repelling sequence 26 shown in FIG. 3B comprises four magnetic components 20. Proceeding from left to right, the first and second magnetic components 20 are arranged such that the component first face 22A and the first magnetic pole 46A of the first magnetic component 20 is oriented towards the component first face 22A and the first magnetic pole 46A of the second magnetic component. Continuing down the repelling sequence 26, the second and third magnetic components 20 are arranged such that the component second face 22B and the second magnetic pole 46B of the second and third magnetic components are oriented towards each other. Lastly, the fourth magnetic component 20 is oriented such that the component first face 22A and the first magnetic pole 46A of the fourth magnetic component 20 is oriented towards the component first face 22A and the first magnetic pole 46A of the third magnetic component 20.

Turning to FIG. 4A while also referring to FIG. 3B, the magnetic fields 40 are represented through magnetic field lines 40L which flow from the first magnetic pole 46A of each magnetic component 20 toward the second magnetic pole 46B. Where an attracting force is exerted between two adjoining magnets, a portion of the magnetic field lines 40L would flow towards the adjoining magnetic poles of opposite polarity. However, when magnetic poles of like polarity are placed in adjoining proximity, the magnetic fields 40 emitted by each magnetic component 20 will repel the magnetic field 40 of the adjacent magnetic component 20. The magnetic field 40 of each magnetic component 20 will be repelled away from the adjacent magnetic component 20, causing the magnetic field lines 40L to flow outwardly away from the magnetic axis 48. The repelling configuration therefore widens and disperses the magnetic fields 40 of the magnetic components 20 within the repelling sequence 26.

Referring to FIG. 4C while also referring to FIG. 1B, FIG. 3B, and FIG. 4A, an exemplary repelling magnet assembly 10 is shown arranged in a loop configuration 19. The retaining member 12 is formed as a circular or ring-like structure which allows the repelling sequence 26 to annularly surround a portion of the wearer's body such as the wrist 66. When in the loop configuration 19, the repelling magnet assembly 10 has a loop interior space 19B through which the wrist 66 or other portion of the body, such as a leg or neck, may extend. The repelling configuration causes the magnetic fields 40 of the magnetic components 20 within the repelling sequence 26 to be directed inwardly in relation to the loop 19 towards the loop interior space 19B, and towards the body of the wearer 60. In one embodiment, the retaining member 12 is formed of a material with sufficient strength to prevent deformation as a result of the repelling forces 44 exerted by the magnetic components 20. For example, the retaining member 12 may be formed of a rigid plastic material or non-ferrous metal which prevents deformation off the loop configuration 19.

Turning to FIG. 3B and FIG. 4B while also referring to FIG. 4C, the repelling force 44 exerted between the adjoining magnetic components 20 may be strengthened by increasing the number of the magnetic components 20 in relation to the length of the retaining member 12. The repelling force 44 is inversely proportional to the length of the separation interval 28. For example, by maintaining the same length of the retaining member 12 but doubling the number of magnetic components 20 within the repelling sequence 26, the separation interval 28 between each of the adjoining magnetic components 20 will be reduced. Furthermore, increasing the number of magnetic components 20 further concentrates additional magnetic fields 40 within the loop interior space 19B. In one example, the separating interval 28 between each pair of adjacent magnetic components 20 may be less than a corresponding thickness of each magnetic component 20 as measured between the component first face 22A and the component second face 22B. The separating intervals 28 may be minimized by maximizing the number of the magnetic components 20, and/or by altering the magnetic strength of each of the magnetic components 20 through the ferromagnetic composition.

In certain embodiments where the retaining member 12 forms a loop configuration 19, the magnetic components 20 within the repelling sequence 26 form a continuous circular sequence whereby each of the magnetic components 20 repels the magnetic components 20 adjacent to it. The number of magnetic components 20 can be maximized by increasing the number of magnetic components 20 until the length of the retaining member 20 can accommodate no further magnetic components 20, and/or until the repelling forces 44 are so great that no further magnetic components 20 may be added.

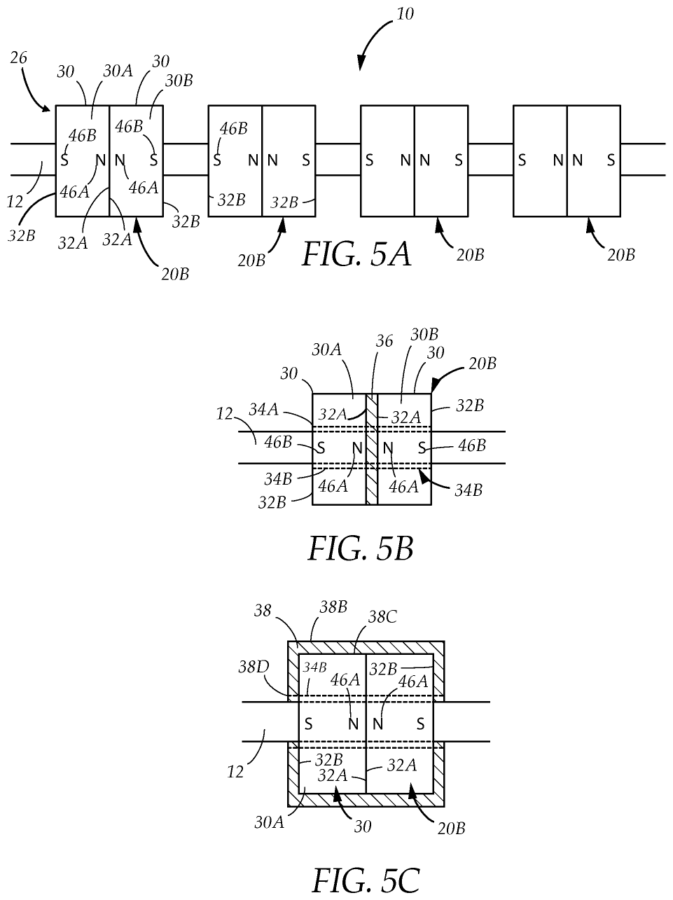

Turning now to FIG. 2B and FIGS. 5A-C while also referring to FIG. 2A. FIG. 3B, and FIG. 4A, in an alternate embodiment, the repelling sequence 26 can be formed using a plurality of alternate magnetic components 20B each comprising two magnetic subcomponents 30, corresponding to a first magnetic subcomponent 30A and a second magnetic subcomponent 30B. Note that each alternate magnetic component 20B may be arranged to form repelling sequences 26 in substantially the same manner as magnetic components 20, in accordance with the principles of the present disclosure.

In one embodiment, each magnetic subcomponent 30 has a subcomponent first face 32A, a distally oriented subcomponent second face 32B, and a subcomponent body 30C which extends therebetween. As with the magnetic components 20, each magnetic subcomponent 30 contains ferromagnetic metals and can be configured in a variety of shapes. For example, each magnetic subcomponent 30 can be formed as a disc, with a cylindrical subcomponent outer surface 32C which extends between the subcomponent first face 32A and the subcomponent second face 32B. Each magnetic subcomponent 30 further has a subcomponent channel opening 34A positioned on each of the subcomponent first and second faces 32A, 32B and a subcomponent channel 34B extending therebetween.

In a preferred embodiment, each magnetic subcomponent 30 has a first magnetic pole 46A oriented with the subcomponent first face 32A and a second magnetic pole 46B oriented with the component second face 32B. Each alternate magnetic component 20B is formed by joining the subcomponent first face 32A of the first magnetic subcomponent 30A to the subcomponent first face 32A of the second magnetic subcomponent 30B. The repelling force 44 exerted between the first and second magnetic subcomponents 30A, 30B can be overcome by using a strong bonding means 36 which maintains the physical contact between the first and second magnetic subcomponents 30A, 30B. The bonding means 36 may be a strong adhesive. Alternatively, the subcomponent first faces 32A of the first and second magnetic subcomponents 30A, 30B may be configured with complementary attachment threads creating a threaded bond therebetween.

In another embodiment, the first and second magnetic subcomponents 30A, 30B are held within a containing shell 38. The containing shell 38 is substantially hollow and has a shell outer surface 38B with a shell cavity 38C formed within. The shell cavity 38C is configured to substantially match the shape and dimensions of the alternate magnetic component 20B, and is sufficiently strong to hold the first and second magnetic subcomponents 30A, 30B in direct contact and prevent the separation thereof. In one example, the alternate magnetic component 20B is formed as a cylinder, and the shell cavity 38C is suitably formed in a cylindrical configuration. The containing shell 38 may be formed using materials such as hard plastic which have sufficient strength to overcome the repelling force 44 without interfering with the magnetic fields 40.

The containing shell 38 further has a pair of shell cavity openings 38D, each of which directly align with the subcomponent channel 34B and subcomponent channel openings 34A of the first and second magnetic subcomponents 30A, 30B to allow the retaining member 12 to pass therethrough.

In a preferred embodiment, the subcomponent second faces 32B and the second magnetic poles 46B of the first and second subcomponents 30A, 30B face outwardly away from the abutting subcomponent first faces 32A. As a result, each alternate magnetic subcomponent 20B will always repel any alternate magnetic subcomponent 20B which is adjacent thereto within the repelling sequence 26.

Each magnetic subcomponent 30 within the alternate magnetic component 20B emits a magnetic field 40, which is then repelled by both the repelling force 44 exerted between the first magnetic poles 46A of the first subcomponent 30A and the second subcomponent 30B, and by the repelling force 44 exerted by the second magnetic pole 46B of each alternate magnetic component 20B adjacent thereto. Furthermore, as the distance between the first magnetic poles 46A of the first and second magnetic subcomponents 30A, 30B is minimal, the strength of the repelling force 44 exerted therebetween is greatly increased, causing an increased widening of the magnetic fields 40 emitted by the alternate magnetic components 20B.

Turning to FIG. 4B, in one embodiment, placing two like magnetic polarities in close proximity through the repelling configuration of the repelling sequence 26, creates a magnetic collision which increases movement of free electrons 42 within the magnetic field 40, and has the potential to alter voltage within cells of the human body, as well as increase pH levels in water.

Turning now to FIGS. 6A-B while also referring to FIG. 3B and FIG. 4C, the retaining member 12 can be placed in a segment configuration 19S which can be substantially linear or arcuate in form. The retaining member 12 in the segment configuration 19S may have a first end 12A and a second end 12B, and the magnetic components 20 may be threaded over the retaining member 12 to be held between the first and second ends 12A, 12B. The first and last magnetic components within the repelling sequence 26 may abut against a retaining end 16 disposed at the first and second ends 12A, 12B of the retaining member 12. The retaining ends 16 have a width greater than the width of the channel opening 24A or channel 34, and thus prevent the repelling forces 44 from increasing the separation intervals 28 between the magnetic components 20 and widening the repelling sequence 26. Alternatively, in certain embodiments, the outermost magnetic components 20 within the repelling sequence 26 may be bonded or adhered to the retaining member 12.

In one embodiment, the loop configuration 19 may allow the retaining member 12 to be selectively opened or closed to facilitate ease of wear and removal. As such, the retaining member 12 is fashioned into circular or ring-like shape, and the first end 12A and the second end 12B are detachable, and are joined together by a fastening means 18 positioned therebetween. In one embodiment, the fastening means 18 may correspond to clasp mechanisms 18C, such as jewelry clasps, which are positioned on the first and second ends 12A, 12B. Note that other fastening means 18 and fasteners may be used, as will be appreciated by a person of ordinary skill in the art in the field of the invention. For example, the fastening means 18 may encompass a variety of detachable fasteners suitable for use with jewelry and other wearable accessories.

In certain embodiments, the repelling magnet assembly 10 may be worn as an attachment to a chain, string, or other suitable hanging means connected to the first and second ends 12A, 12B of the retaining member 12. In other embodiments, the repelling magnet assembly 10 may be held close to the body through integration into articles of clothing.

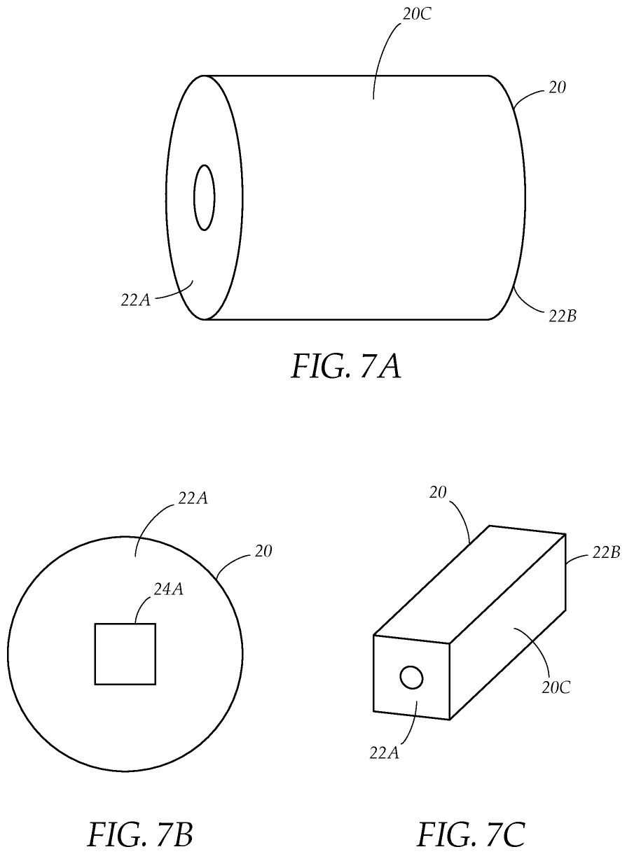

Turning to FIGS. 7A-C while also referring to FIGS. 3A-B, the magnetic components 20 may be formed into various shapes such as elongated cylinders or as rectangular prisms, without altering the arrangement of magnetic poles as described herein. Furthermore, in certain embodiments, the retaining member 12 and the channel opening 24A and/or channel 24B may be configured with alternative cross sectional shapes. For example, the channel opening 24A, the channel 24B, and the retaining member 12 may have a round cross sectional shape, or a square or polygonal cross sectional shape.

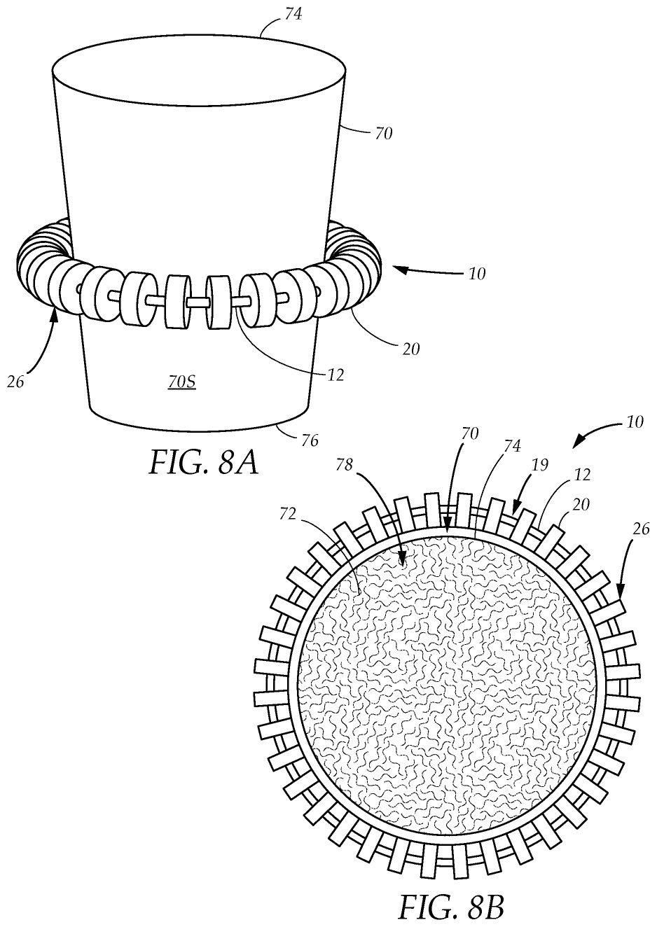

Turning now to FIGS. 8A-B while also referring to FIG. 4C, in an alternative embodiment, the repelling magnet assembly 10 in the loop configuration 19 may be arranged to surround a container 70 for storing a liquid 78, such as a cup or bottle. The container 70 may have a container opening 74, a container base 76, and a container surface 70S extending therebetween. The container 70 further has a container interior 72 for holding the liquid 78, which is accessible via the container opening 74. The repelling magnet assembly 10 may be positioned between the container opening 74 and the container base 76 to surround the container surface 70S. The magnetic fields 40 emitted by the repelling sequence 26 extend inwardly into the interior space 19S to pass through the liquid 78, thus allowing the magnetic fields 40 to increase the pH level of the liquid 78.

It is understood that when an element is referred hereinabove as being “on” another element, it can be directly on the other element or intervening elements may be present therebetween. In contrast, when an element is referred to as being “directly on” another element, there are no intervening elements present.

Moreover, any components or materials can be formed from a same, structurally continuous piece or separately fabricated and connected.

It is further understood that, although ordinal terms, such as, “first,” “second,” “third,” are used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

Spatially relative terms, such as “beneath,” “below,” “lower,” “above,” “upper” and the like, are used herein for ease of description to describe one element or feature's relationship to another element(s) or feature(s) as illustrated in the figures. It is understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

Example embodiments are described herein with reference to cross section illustrations that are schematic illustrations of idealized embodiments. As such, variations from the shapes of the illustrations as a result, for example, of manufacturing techniques and/or tolerances, are to be expected. Thus, example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

In conclusion, herein is presented a repelling magnet assembly. The disclosure is illustrated by example in the drawing figures, and throughout the written description. It should be understood that numerous variations are possible, while adhering to the inventive concept. Such variations are contemplated as being a part of the present disclosure.

What is claimed is:

1. A repelling magnet assembly, comprising:
a plurality of magnetic components, each magnetic component has a channel and projects a magnetic field;
a retaining member which passes through the channel of each of the magnetic components; and
a repelling sequence formed from the plurality of magnetic components disposed linearly along the retaining member, each of the magnetic components within the repelling sequence exerts a repelling force against the magnetic components which are adjacent to said magnetic component.

2. The repelling magnet assembly as described in claim 1, wherein the repelling forces exerted by the plurality of magnetic components create a separation interval between each of the magnetic components.

3. The repelling magnet assembly described in claim 2, wherein:
each magnetic component has a first magnetic pole, and a second magnetic pole oriented distally relative to the first magnetic pole, the repelling force exerted between each of the magnetic components is produced by orienting the first magnetic pole towards another of the first magnetic poles or by orienting the second magnetic pole towards another of the second magnetic poles.

4. The repelling magnet assembly described in claim 3, wherein:
each magnetic component has a magnetic axis extending coaxially with the channel, and the repelling force exerted upon each magnetic component repels the magnetic field of said magnetic component outwardly away from the magnetic axis.

5. The repelling magnet assembly described in claim 4, wherein:
the retaining member and the channel of each of the magnetic components each have a width, and the width of the retaining member is less than the width of the channels of the magnetic components.

6. The repelling magnet assembly described in claim 5, wherein:
the retaining member is configured as a loop, allowing the repelling magnet assembly to be worn by a wearer with a body such that the retaining member surrounds a portion of the body.

7. The repelling magnet assembly described in claim 6, wherein:
the loop of the retaining member has a fastener adapted to allow the loop to be selectively closed or opened to facilitate wearing or removal by the wearer.

8. The repelling magnet assembly described in claim 5, wherein:
the retaining member is configured as a segment, the retaining member has a first end and a second end, and the magnetic components within the repelling sequence are disposed between the first end and the second end.

9. The repelling magnet assembly described in claim 2, wherein:
each of the magnetic components comprises two magnetic subcomponents including a first magnetic subcomponent and a second magnetic subcomponent, each of the magnetic subcomponents has a subcomponent first face, a subcomponent second face oriented distally relative to the subcomponent second face, a first magnetic pole oriented with the subcomponent first face, and a second magnetic pole oriented with the subcomponent second face, the subcomponent first face of the first magnetic subcomponent is directly attached to the subcomponent first face of the second magnetic subcomponent, causing the repelling force to be generated between the first magnetic poles of the first magnetic subcomponent and the second magnetic subcomponent; and
the second magnetic poles of the first magnetic subcomponent and the second magnetic subcomponent face outwardly away from the subcomponent first faces, and generate the repelling force between each magnetic component and the adjacent magnetic components within the repelling sequence.

10. The repelling magnet assembly described in claim 9, wherein:
the repelling force exerted between the subcomponent first face of the first magnetic subcomponent and the subcomponent first face of the second magnetic subcomponent is overcome by a bonding means which prevents the magnetic subcomponent from separating therebetween.

11. The repelling magnet assembly described in claim 9, wherein:
each magnetic component is encased within a containing shell which overcomes the repelling force exerted between the subcomponent first face of the first magnetic subcomponent and the subcomponent first face of the second magnetic subcomponent, and prevents the magnetic component from separating therebetween.

12. The repelling magnet assembly described in claim 11, wherein:
the containing shell has a shell outer surface, a shell cavity for containing the magnetic component, and pair of shell channel openings aligned with the channel of the magnetic component and which allow the retaining member to pass therethrough.

13. A method for exposing a wearer having a body to magnetism, comprising the steps of:
providing a plurality of magnetic components, each magnetic component has a channel and projects a magnetic field;
providing a retaining member adapted to pass through the channels of each of the magnetic components;
forming a repelling sequence comprising the plurality of magnetic components disposed linearly along the retaining member;
exerting a repelling force by each of the magnetic components against each magnetic component adjacent to said magnetic component;
placing the repelling sequence in close proximity to the body of the wearer; and
exposing the body to the magnetic fields produced by the magnetic components.

14. The method for exposing a wearer having a body to magnetism as recited in claim 13, wherein:
each magnetic component has a magnetic axis extending coaxially with the channel; and
the step of exerting a repelling force is followed by the step of repelling the magnetic field of each of the magnetic components outwardly away from the magnetic axis by the repelling force.

15. A method for exposing a wearer having a body to magnetism as recited in claim 14, wherein:
the step of exerting a repelling force further comprises creating a separation interval between each of the magnetic components.

16. A method for exposing a wearer having a body to magnetism as recited in claim 15, wherein:
the step of forming a repelling sequence is preceded by the step of forming the retaining member in a loop configuration having a loop interior space; and
the step of placing the repelling sequence in close proximity to the body of the wearer further comprises surrounding a portion of the body within the loop interior space by the retaining member.

17. A method for exposing a wearer having a body to magnetism as recited in claim 16, wherein:
the step of repelling the magnetic field of each of the magnetic components further comprises repelling the magnetic field of each of the magnetic components towards the loop interior space.
